(19) United States
(12) Patent Application Publication
(10) Pub. No.: US 2022/0344041 A1
(43) Pub. Date: Oct. 27, 2022

(54) INFORMATION PROCESSING DEVICE, INFORMATION PROCESSING METHOD, AND PROGRAM

(71) Applicant: SONY GROUP CORPORATION, TOKYO (JP)

(72) Inventors: MAKI SHIGYO, TOKYO (JP); MASANORI KATSU, TOKYO (JP)

(21) Appl. No.: 17/640,748

(22) PCT Filed: Jul. 15, 2020
(86) PCT No.: PCT/JP2020/027461

§ 371 Date: Mar. 4, 2022

(30) Foreign Application Priority Data
Sep. 13, 2019 (JP) 2019-167008

(51) Int. Cl. G16H 40/63; G16H 20/30; G16H 20/70

(52) U.S. Cl. CPC G16H 40/63; G16H 20/30; G16H 20/70

(57) ABSTRACT

A measurement device 20 acquires biological information of a user and transmits the biological information to a terminal device 30-1. The terminal device transmits the biological information to a server 40. The server 40 calculates physical power and mental power using the biological information of the user. Furthermore, the server 40 generates activity support information for the user on the basis of the physical power and the mental power that are calculated, and transmits the activity support information to the terminal device 30-1. The terminal device 30-1 notifies the user of the activity support information supplied from the server 40 by an image or a sound. Therefore, the user can determine a type of activity and a time length that are possible with his/her remaining powers, and can perform an activity according to his/her remaining powers.

### TECHNICAL FIELD

The present technology relates to an information processing device, an information processing method, and a program, and makes it possible to determine a state of a user from multiple sides and notify the user of possible activities and activity time lengths.

### BACKGROUND ART

Conventionally, there has been proposed notification of stamina information or the like indicating a power of a user on the basis of biological information or the like of the user. For example, in Patent Document 1, stamina information of a user is output in a manner that the user can easily grasp the stamina information.

### CITATION LIST

### Patent Document

Patent Document 1: Japanese Patent Application Laid-Open No. 2017-169732

### SUMMARY OF THE INVENTION

### Problems to be Solved by the Invention

Meanwhile, Patent Document 1 discloses that first stamina information indicating the endurance (stamina) of a user is indicated, and at least one of second stamina information at an environment situation different from that of the first stamina information, third stamina information of another user, advice information or alert information for an activity of the user, and exercise continuity information of the user is included. However, even if the user has endurance, it is difficult to perform an activity without the user's motivation, and there may be a case where advice information or alert information suitable for the user cannot be output even by using the first stamina information, the second stamina information, and the third stamina information.

Therefore, an object of this technology is to provide an information processing device, an information processing method, and a program capable of determining a state of a user from multiple sides and notifying the user of possible activities and activity time lengths.

### Solutions to Problems

A first aspect of the present technology is

an information processing device including:

a power calculation unit configured to calculate a physical power and a mental power by using biological information of a user; and

an information generation unit configured to generate activity support information for the user on the basis of the physical power and the mental power calculated by the power calculation unit.

In the present technology, the power calculation unit calculates a physical power and a mental power by using biological information of a user, and the information generation unit generates activity support information indicating an activity and an activity time length that are possible on the basis of the physical power and the mental power that are calculated, or an activity capable of recovering the physical power or the mental power. When any activity is selected from the activity that is possible, the information generation unit indicates, in the activity support information, an activity and an activity time length that are newly possible on the basis of the physical power and the mental power after the selected activity is performed. Furthermore, the information generation unit calculates an activity time length using the activity environment information. Furthermore, the information generation unit generates activity support information for the user and another person different from the user on the basis of the minimum value among the physical powers of the user and the other person different from the user and the minimum value among the mental powers of the user and the other person different from the user. Note that the activity support information may include information indicating the person corresponding to the minimum value.

The power calculation unit calculates the physical power using an index related to a physical activity based on the biological information of the user, and calculates the mental power using an index related to a mental activity based on the biological information. Furthermore, the power calculation unit calculates initial values of the physical power and the mental power by using biological information when the user is in a best condition and the biological information of the user acquired at a start timing. Moreover, the power calculation unit updates the physical power and the mental power using biological information of the user acquired after the start timing.

In the activity support information, temporal changes of the physical power and the mental power ae indicated in a two-dimensional indication or a ratio indication, or as changes of an attribute in a temporal progress status indication. Furthermore, the activity support information may indicate a possible activity on the basis of the physical power and the mental power using a map. For example, in the activity support information, a spot and a route of an activity that is possible on the basis of the physical power and the mental power may be indicated on the map, and notification of the physical power and the mental power at a specified position of the route may be performed. Furthermore, in the activity support information, notification of the route may be performed with an icon moving on the map. Furthermore, the activity support information may include movement route information and instruction information from a managing person who manages the user, and may include a list of the physical powers and the mental powers of management target users calculated by the power calculation unit, or power history information indicating temporal changes of the physical power and the mental power of at least one of the management target users specified by the managing person.

A second aspect of the present technology is

an information processing method including:

calculating, by a power calculation unit, a physical power and a mental power by using biological information of a user; and

generating, by an information generation unit, activity support information for the user on the basis of the physical power and the mental power calculated by the power calculation unit.

A third aspect of the present technology is

a program for causing a computer to perform activity support for a user by causing the computer to:

calculate a physical power and a mental power by using biological information of the user; and

generate activity support information for the user on the basis of the physical power and the mental power that are calculated.

Note that the program of the present technology is, for example, a program that can be provided, in a computer-readable format to a general-purpose computer capable of executing various program codes, by a storage medium, or a communication medium, and examples of the storage medium include an optical disk, a magnetic disk, or a semiconductor memory, and examples of the communication medium include a communication medium such as a network. By providing such a program in a computer-readable format, processing according to the program is implemented on the computer.

### BRIEF DESCRIPTION OF DRAWINGS

FIG. 1 is a diagram illustrating a configuration example of an information processing system.

FIG. 2 is a diagram illustrating a configuration example of a measurement device.

FIG. 3 is a diagram illustrating a configuration example of a terminal device.

FIG. 4 is a diagram illustrating an example of a configuration of a server.

FIG. 5 is a flowchart illustrating an example of an operation of the information processing system.

FIG. 6 is a diagram illustrating an example of a basic information setting screen in the initial setting processing.

FIG. 7 is a diagram illustrating an example of an optimum value setting screen in the initial setting processing.

FIG. 8 is a diagram illustrating an example of the initial value input screen.

FIG. 9 is a diagram for describing a sleep time length score.

FIG. 10 is a diagram for describing blood pressure scores.

FIG. 11 is a diagram illustrating an example of an initial value score display screen.

FIG. 12 is a diagram illustrating an example of a power initial value notification screen.

FIG. 13 is a diagram illustrating an example of the power change amount of the physical power according to the exercise strength.

FIG. 14 is a diagram illustrating an example of the power change amount of the mental power according to stress.

FIG. 15 is a diagram illustrating an example of a remaining power notification.

FIG. 16 is a diagram illustrating an example of a notification of activity support information.

FIG. 17 is a diagram illustrating an example of a notification of activity support information indicating an activity and activity time length that are further possible according to the selected activity.

FIG. 18 is a diagram illustrating an example of a notification of activity support information indicating a possible activity range in a case where walking is selected as an activity.

FIG. 19 is a diagram illustrating another example of a notification of activity support information indicating a possible activity range.

FIG. 20 is a diagram illustrating an example of correction of the exercise strength based on activity environment information.

FIG. 21 is a diagram illustrating an example of a remaining power notification of a user A and a user B traveling together.

FIG. 22 is a diagram illustrating an example of a notification of activity support information generated on the basis of the remaining powers using map information.

FIG. 23 is a diagram illustrating an example of the operation of changing the activity support information after the deletion of stopping by to some spots.

FIG. 24 is a diagram illustrating an example of an operation of changing the activity support information after the moving means is selected.

FIG. 25 is a diagram illustrating an example of a change operation in a case where a spot where the powers can be recovered is selectable.

FIG. 26 is a diagram illustrating an example of a notification of activity support information in a case where a user goes shopping.

FIG. 27 is a diagram illustrating an example of a notification of activity support information to a managed person who has been requested to shop or the like.

FIG. 28 is a diagram illustrating an example of a notification of the remaining power list information.

FIG. 29 is a diagram illustrating an example of the power history information of a specified employee.

FIG. 30 is a diagram illustrating an example of a notification of activity support information to a specified employee.

### MODE FOR CARRYING OUT THE INVENTION

Hereinafter, modes for carrying out the present technology will be described. Note that the description will be given in the following order.

1. Information Processing System

2. Configuration of Embodiment

3. Operation of Embodiment

4. Application Example

4-1. First Application Example

4-2. Second Application Example

4-3. Third Application Example

4-4. Fourth Application Example

### 1. INFORMATION PROCESSING SYSTEM

In the present technology, the physical power and the mental power are calculated using the biological information of a user, and activity support information is generated on the basis of the physical power and the mental power that are calculated and provided to the user. The biological information of the user is acquired by a measurement device worn by the user.

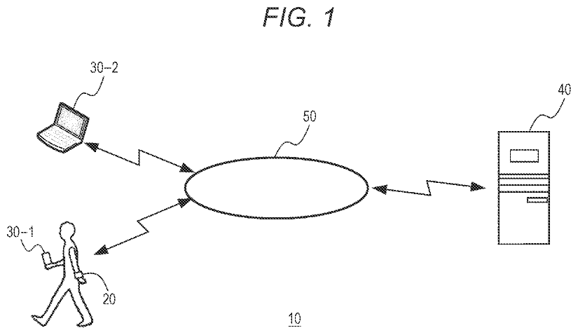

FIG. 1 illustrates a configuration example of an information processing system using the present technology. An information processing system 10 includes a measurement device 20, a terminal device 30-1 (30-2), and a server 40. The measurement device 20 or the terminal device 30-1 (30-2) is connected to the server 40 via a network 50. Furthermore, the measurement device 20 may be configured to enable wireless communication with the terminal device 30-1 (30-2).

The measurement device 20 acquires the biological information of a user. A power calculation unit that calculates the physical power and the mental power using the biological information of the user and an information generation unit that generates the activity support information for the user on the basis of the physical power and the mental power calculated by the power calculation unit may be provided in either the terminal device 30-1 (30-2) or the server 40, or may be provided in the measurement device 20. The terminal device 30-1 (30-2) notifies the user of the activity support information generated by the information generation unit using an image or a sound.

In a case where the terminal device 30-1 (30-2) includes the power calculation unit and the information generation unit, the measurement device 20 transmits the acquired biological information to the terminal device 30-1 (30-2). Furthermore, the terminal device 30-1 (30-2) acquires information necessary for generating the activity support information such as map information to be described later, remaining powers of another person, and the like from the server 40.

In a case where the server 40 includes the power calculation unit and the information generation unit, the measurement device 20 transmits the acquired biological information to the server 40 via the terminal device 30-1 (30-2) or to the server 40 without interposing the terminal device 30-1 (30-2) therebetween. Furthermore, the terminal device 30-1 (30-2) transmits, to the server 40, information input by the user and used for generating the activity support information, and the like. Hereinafter, a configuration and an operation in a case where the power calculation unit and the information generation unit are provided in the server 40 will be described.

### 2. CONFIGURATION OF EMBODIMENT

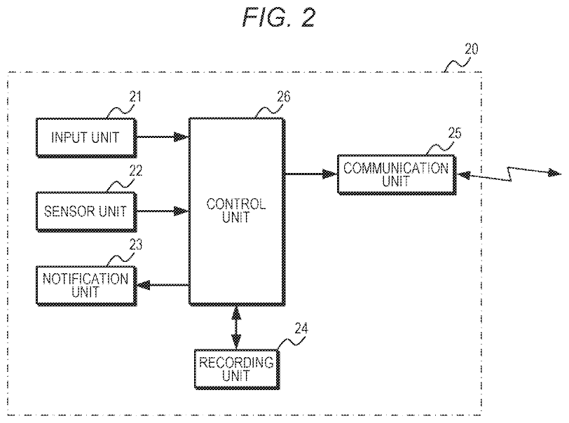

FIG. 2 illustrates a configuration example of a measurement device. The measurement device 20 includes an input unit 21, a sensor unit 22, a notification unit 23, a recording unit 24, a communication unit 25, and a control unit 26.

The input unit 21 generates an operation signal corresponding to a user operation such as operation setting and outputs the operation signal to the control unit 26.

The sensor unit 22 acquires biological information of the user such as information indicating a pulse rate (heart rate), a body temperature, blood pressures, a blood flow, an activity time length (sleep time length), an activity state (exercise state), and the like of the user. The sensor unit 22 uses a pulse wave sensor in a case of acquiring pulse wave information indicating a pulse rate (heart rate) or the like as biological information. The pulse wave sensor includes, for example, a photoelectric sensor including a light emitting unit and a light receiving unit, an ultrasonic sensor, or the like. Furthermore, the sensor unit 22 uses a temperature sensor in a case of acquiring information indicating a body temperature, and a blood pressure and blood flow sensor or the like in a case of acquiring information indicating blood pressures or a blood flow. Furthermore, as the sensor unit 22, a respiration monitor or the like is used in a case of acquiring information indicating a respiratory rate. Moreover, as the sensor unit 22, a body motion sensor is used in a case of acquiring information indicating an activity time length and an activity state. The body motion sensor includes, for example, a gyro sensor, a six-axis sensor including a three-axis acceleration sensor and a three-axis gyro sensor, a positioning sensor, and the like, and measures a moving distance, the number of steps, a stride length, a moving time length, a moving speed, an acceleration or a variation in acceleration, a momentum, activity content, an altitude difference per unit time length, and the like of the user. Note that the sensor unit 22 may use a camera capable of imaging a user such that the concentration level and the drowsiness level can be determined from the expression of the user or the like.

The notification unit 23 includes a display element, and performs display related to an operation of operation setting of the sensor unit 22, display indicating an operation setting state, display of biological information acquired by the sensor unit 22, and the like. Furthermore, the recording unit 24 records the biological information acquired by the sensor unit 22.

The communication unit 25 performs wireless communication with the terminal device 30-1 (30-2) and the server 40 to transmit the biological information acquired by the sensor unit 22, and the biological information acquired by the sensor unit 22 and recorded in the recording unit 24 to the terminal device 30-1 (30-2) and the server 40.

The control unit 26 controls the operation of each unit of the measurement device 20 on the basis of an operation signal from the input unit 21 to acquire the biological information of the user and transmit the acquired biological information from the communication unit 25 to the terminal device 30-1 (30-2) and the server 40. Note that the control unit 26 may control the operation of each unit of the measurement device 20 on the basis of an operation signal received from the terminal device 30-1 (30-2) via the communication unit 25.

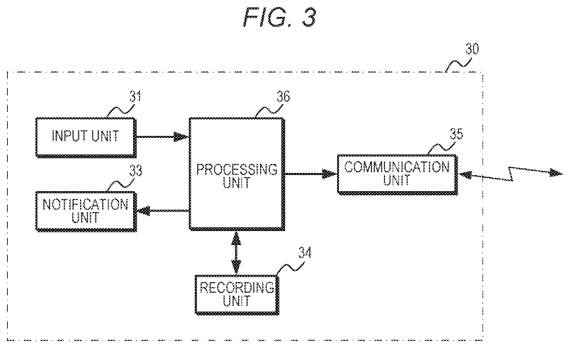

FIG. 3 illustrates a configuration example of a terminal device. For example, the terminal device 30-1 is a portable terminal device such as a smartphone. Furthermore, the terminal device 30-2 is a notebook computer device or a desktop computer device. Hereinafter, the terminal device 30-1 and the terminal device 30-2 will be described as a terminal device 30.

The terminal device 30 includes an input unit 31, a notification unit 33, a recording unit 34, a communication unit 35, and a processing unit 36.

The input unit 31 generates an operation signal corresponding to a user operation such as operation setting and outputs the operation signal to the processing unit 36.

The notification unit 33 includes a display element or a sound output element, and notifies the user of activity support information received from the server 40 via the communication unit 35 to be described later or activity support information generated by the processing unit 36.

The recording unit 34 records various kinds of information including, for example, activity support information received from the server 40, or activity support information generated by the processing unit 36.

The communication unit 35 performs wireless communication with the measurement device 20 to receive biological information transmitted from the sensor unit 22. Furthermore, the communication unit 35 communicates with the server 40 via the network 50 to transmit biological information to the server 40. Furthermore, the communication unit 35 receives activity support information transmitted from the server 40 and outputs the activity support information to the processing unit 36. Moreover, the communication unit 35 may communicate with a terminal device 30 of another user.

The processing unit 36 controls an operation of each unit of the terminal device 30 on the basis of an operation signal from the input unit 31. For example, the processing unit 36 transmits, to the server 40, biological information supplied from the measurement device 20, information input by a user from the input unit 31 or the like and used for generating activity support information, and the like. Furthermore, the processing unit 36 performs processing of notifying the user of activity support information, which is supplied from the server 40, from the notification unit 33 by an image or a sound. Note that in a case where the terminal device 30 generates activity support information, the functions of the power calculation unit and the information generation unit are provided in the processing unit 36.

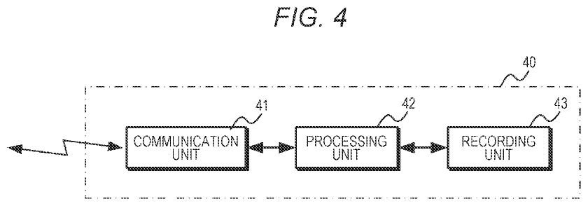

FIG. 4 illustrates a configuration example of a server. The server 40 includes a communication unit 41, a processing unit 42, and a recording unit 43. Note that the server 40 may be either an on-premises server or a cloud server.

The communication unit 41 communicates with the measurement device 20 and the terminal device 30 via the network 50, and outputs, to the processing unit 42, biological information acquired by the measurement device 20. Furthermore, the communication unit 41 outputs, to the terminal device 30, activity support information generated by the processing unit 42. Moreover, the communication unit 41 may communicate with another server to acquire information about weather, temperature, humidity, and the like.

The processing unit 42 has functions of the power calculation unit and the information generation unit. The processing unit 42 calculates the physical power and the mental power of a user. The physical power is a power related to a physical element, and is calculated using an index that changes in conjunction with a change in muscle strength, agility, endurance, resistance to physical stress, or the like. The mental power is a power related to a mental element, and is calculated using an index that changes in conjunction with a change in concentration, judgment, resistance to mental stress, or the like. The processing unit 42 calculates the physical power using, for example, an index related to blood circulation, an index related to respiration, or the like based on the biological information of a user acquired by the measurement device 20, and calculates the mental power using, for example, an index related to concentration or an index related to mental stress based on the ecological information of the user. Note that the above-described indices used to calculate the physical power and the mental power are examples, and other indices may be used, and a common index may be used to calculate the physical power and the mental power.

The processing unit 42 generates activity support information for a user on the basis of the physical power and the mental power that are calculated. Furthermore, the processing unit 42 uses map information recorded in the recording unit 43 to generate activity support information indicating possible activities using an actual map or as a virtual map. Moreover, in a case where the biological information includes information indicating the current position of the user, the processing unit 42 generates activity support information based on the current position of the user. The processing unit 42 causes the generated activity support information to be output from the communication unit 41 to the terminal device 30.

Furthermore, the processing unit 42 generates activity support information corresponding to a group including a plurality of users. For example, the processing unit 42 may generate activity support information for the user and another person different from the user on the basis of the physical power and the mental power of the user and the other person.

The recording unit 43 records biological information for each user to enable generation of activity support information corresponding to a group including a plurality of users. Furthermore, the recording unit 43 records, in advance, map information indicating positions of various facilities, distances to the facilities, parameter information for calculating a power consumption amount and a power recovery amount, and the like so that the processing unit 42 can generate activity support information using the map information. Furthermore, the recording unit 43 may record preference, past activity history, and the like of a user for each user.

### 3. OPERATION OF EMBODIMENT

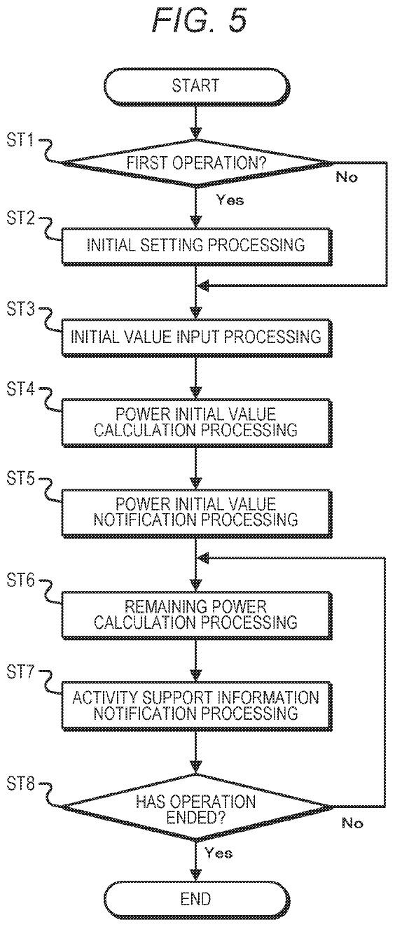

Next, an operation of the information processing system 10 will be described. FIG. 5 is a flowchart illustrating an example of an operation of the information processing system. Note that details of each step will be described later.

In step ST1, the information processing system determines whether the information processing system is operated for the first time. In the information processing system 10, in a case where the system is used for the first time, the processing proceeds to step ST2, and in a case where the system has been used already, the processing proceeds to step ST3.

In step ST2, the information processing system performs initial setting processing. The information processing system 10 performs setting of basic information of a user and setting of an optimum value regarding biological information and the like. For example, in a case where, for example, the function of notification of activity support information is first used, the terminal device 30 displays a basic information setting screen on the notification unit 33, and accepts basic information, for example, age, sex, weight, and the like input by a user operation on the input unit 31.

Furthermore, in a case where, for example, the function of notification of activity support information is first used or in a case where an optimum value resetting operation is performed, the terminal device 30 displays an optimum value setting screen on the notification unit 33, and accepts optimum value information input by a user operation on the input unit 31, and the optimum value information is, for example, the body temperature, the blood pressures, and the like during an optimum sleep time length or when the user is in optimum condition. Note that the optimum value may be automatically set on the basis of the basic information of the user. The terminal device 30 transmits the set basic information and optimum value information to the server 40, and the processing proceeds to step ST3.

In step ST3, the information processing system performs initial value input processing. The terminal device 30 displays an initial value setting screen on the notification unit 33, and accepts, as an initial value, biological information or the like at activity reference timing input by a user operation on the input unit 31. Furthermore, the measurement device 20 may acquire biological information at the activity reference timing, for example, the morning timing, and the terminal device 30 may use, as the initial value, the biological information or the like at the activity reference timing acquired by the measurement device 20. The terminal device 30 transmits the initial value to the server 40, and the processing proceeds to step ST4. Note that the measurement device 20 may transmit, as an initial value, the biological information acquired at the activity reference timing to the server 40.

In step ST4, the information processing system performs power initial value calculation processing. The server 40 of the information processing system 10 calculates a physical power initial value and a mental power initial value for each user using the biological information or the like at activity reference timing that is supplied from the measurement device 20 or the terminal device 30, and the processing proceeds to step ST5.

In step ST5, the information processing system performs power initial value notification processing. The server 40 of the information processing system 10 transmits the calculated power initial values to the terminal device 30 of the corresponding user. The terminal device 30 receives the power initial values transmitted from the server 40, and notifies the user of the power initial values from the notification unit 33 by an image or a sound, and the processing proceeds to step ST6.

In step ST6, the information processing system performs remaining power calculation processing. The measurement device 20 of the information processing system 10 acquires biological information of a user and transmits the acquired biological information to the server 40 or to the server 40 via the terminal device 30. Furthermore, the terminal device 30 accepts, on the input unit 31, user input information related to changes in powers and transmits the user input information to the server 40. The processing unit 42 of the server 40 calculates the remaining powers for each user on the basis of the power initial values calculated in step ST4, the biological information acquired by the measurement device 20 thereafter, and the user input information input on the terminal device 30, and the like, and the processing proceeds to step ST7.

In step ST7, the information processing system performs activity support information notification processing. The server 40 of the information processing system 10 generates activity support information on the basis of the remaining powers calculated in step ST6, or on the basis of the remaining powers, the map information recorded in the recording unit 43, and the remaining powers of another person, and then transmits the activity support information to the terminal device 30 of the user. The terminal device 30 receives the activity support information transmitted from the server 40, and notifies the user of an activity, an activity time length, and the like that are possible from the notification unit 33 by an image or a sound, and the processing proceeds to step ST8.

In step ST8, the information processing system determines whether the operation has ended. For example, in a case where the terminal device 30 determines that the user has gone to bed at night on the basis of biological information or the like, or in a case where the terminal device 30 determines that the user has performed an end operation, the information processing system 10 determines that the operation has ended. Furthermore, in a case where the information processing system 10 determines that the user is active, the information processing system 10 returns to step ST6 and continues to perform the remaining power calculation processing.

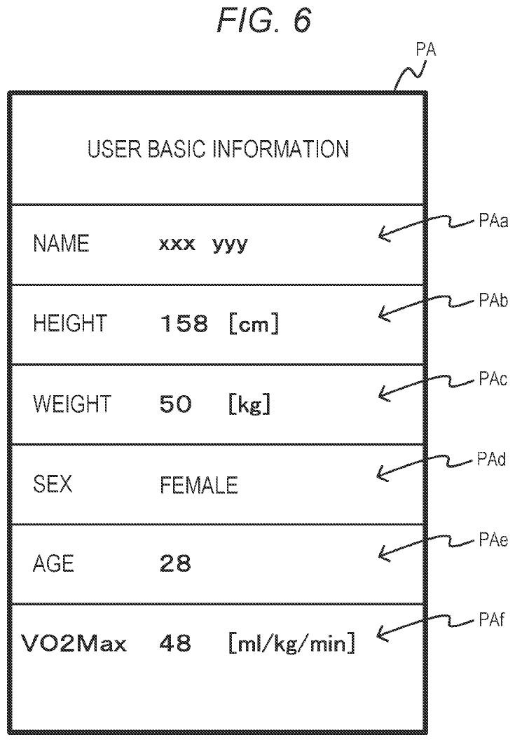

Next, an example of a specific operation of each type of processing will be described. In the initial setting processing, for example, an operation screen for the initial setting processing is displayed on a screen of the notification unit 33 in the terminal device 30. FIG. 6 illustrates an example of a basic information setting screen in the initial setting processing. A basic information setting screen PA is configured to allow a user to input a name PAa, a height PAb, a weight PAc, a sex PAd, an age PAe, a maximum oxygen uptake (VO2Max) PAf, and the like as basic information. The input unit 31 accepts a user input operation for an item indicated on the basic setting screen. The processing unit 36 transmits basic information input from the input unit 31 to the server 40 via the communication unit 35.

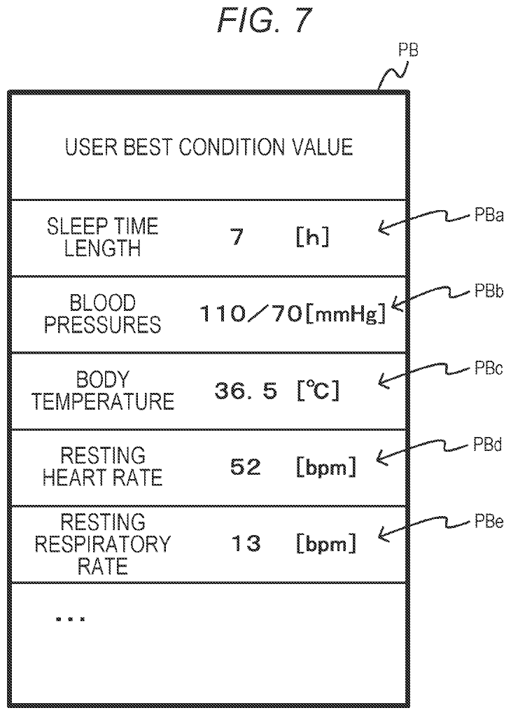

FIG. 7 illustrates an example of an optimum value setting screen in the initial setting processing. An optimum value setting screen PB is configured to allow a user to input a sleep time length PBa, blood pressures PBb, a body temperatures PBc, a resting heart rate PBd, a resting respiratory rate PBe, and the like as optimum values. The input unit 31 accepts a user input operation for an item indicated on the optimum value setting screen. The processing unit 36 transmits optimum values input from the input unit 31 to the server 40 via the communication unit 35. Note that as the optimum values, standard values set in advance on the basis of the basic information or the like may be used.

Next, in the initial value input processing, the measurement device acquires the biological information at the activity reference timing. Alternatively, for example, an initial value input screen that is the operation screen in a case where the initial value input processing is performed is displayed on the screen of the notification unit 33 in the terminal device 30 to allow input of the initial value.

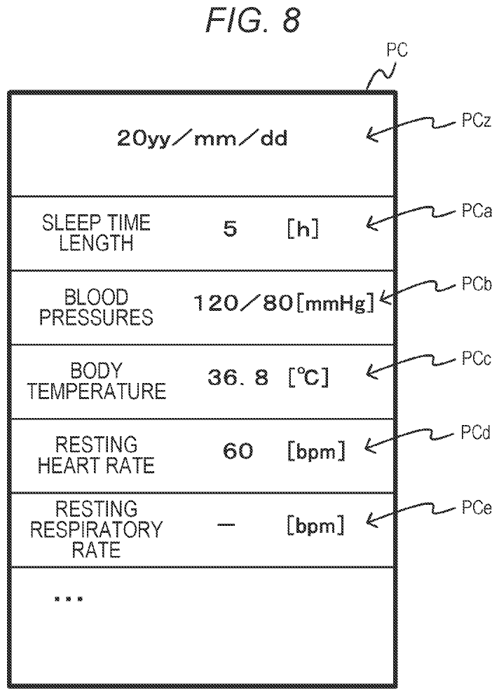

FIG. 8 illustrates an example of the initial value input screen. Similarly to the optimum value setting screen, on an initial value input screen PC, items such as a sleep time length PCa, blood pressures PCb, a body temperature PCc, a resting heart rate PCd, a resting respiratory rate PCe, and the like are indicated. Note that an indication PCz indicates an initial value of the date. The processing unit 36 of the terminal device 30 uses, for example, a measurement value acquired by the measurement device 20 at the activity reference timing as an initial value. Furthermore, the processing unit 36 accepts an initial value input by a user from the input unit 31 for information that has not been acquired by the measurement device 20. The processing unit 36 transmits the initial value acquired by the measurement device 20 and the initial value input from the input unit 31 to the server 40 via the communication unit 35.

Furthermore, the initial value input screen may accept input not only for items similar to the optimum value setting screen but also for conditions of a user, for example, mood (motivation), existence/non-existence of injury, a location and a degree of injury, and the like.

Furthermore, as the input result of the initial value, not only the initial value, but also a ratio to the optimum value (hereinafter, also referred to as a score) may be displayed. The scores may be obtained using, for example, a preset function, or may be obtained using a table or the like.

In the power initial value calculation processing, the physical power is calculated using an index related to a physical activity to calculate the initial value. Specifically, using the initial value input in the initial value input processing, a physical power initial value SRpO is calculated on the basis of, for example, Mathematical Expression (1).

[Mathematical Expression 1]

SRₚ₀=αₚ*SCₚ₁+βₚ*SCₚ₂+γₚ*SCₚ₃+ . . . +εₚ (1)

αₚ+βₚ+γₚ+ . . . ≤(100−εₚ) (2)

0≤(SCₚ₁,SCₚ₂,SCₚ₃, . . . ,)≤1 (3)

0≤εₚ≤100 (4)

In Mathematical Expression (1), a score SCp1 is, for example, an index related to the sleep time length, a score SCp2 is, for example, an index related to the blood circulation, and a score SCp3 is, for example, an index related to the body temperature. The scores may be obtained using a preset function, or may be obtained using a table or the like.

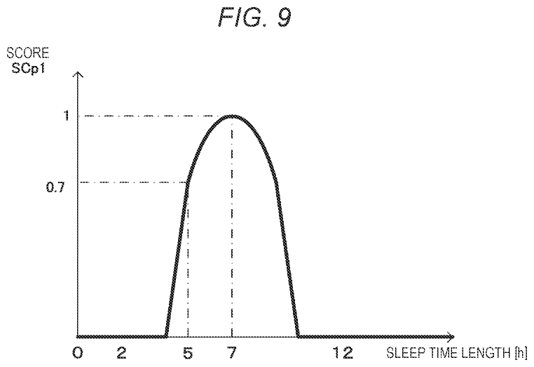

FIG. 9 is a diagram for describing a sleep time length score. In calculation of a score of the sleep time length, a function Fp1 that is set in advance to make the score SCp1 have “1”, which is the maximum value, when the sleep time length is the optimum value and to make the score SCp1 have a lower value when the sleep time length is shorter than the optimum value or longer than the optimum value is used. The server 40 calculates a score for the initial value of the sleep time length using the function Fp1.

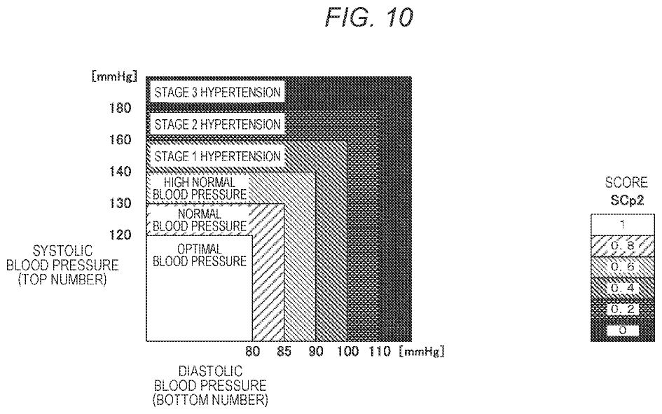

FIG. 10 is a diagram for describing a blood pressure score. For the blood pressures, classification of blood pressure values is performed by making a table defining systolic blood pressures and diastolic blood pressures, and a score is set according to the classification. For example, when the blood pressures fall within a range of the optimal blood pressures, the score SCp2 is set to the maximum value “1”, and when the blood pressures fall within the range of the normal blood pressures, the score SCp2 is set to “0.8”. Furthermore, the score is made smaller as the blood pressure values are classified to be higher, and the score SCp2 is set to “0.1” which is a minimum value when the blood pressures are stage 3 hypertension. Furthermore, the server 40 may calculate a score for the respiratory rate in a manner similar to, for example, calculation of a score for the sleep time length and use the score as an index related to respiration.

The coefficients αp, βp, and γp indicate the weights of the respective scores, and satisfy the condition of Mathematical Expression (2). Furthermore, the scores SCp1, SCp2, SCp3, . . . satisfy the condition of Mathematical Expression (3) as described above, and the offset εp is set in advance to satisfy the condition of Mathematical Expression (4). Note that in the power initial value calculation processing, a coefficient related to an index of an item for which no information has been input is set to “0”, and the coefficients of the other items are set so as to satisfy the condition of Mathematical Expression (2).

Furthermore, in the power initial value calculation processing, the mental power is calculated using an index related to a mental activity to calculate the initial value. Specifically, using the initial value input in the initial value input processing, a mental power initial value SRm0 is calculated on the basis of, for example, Mathematical Expression (5).

[Mathematical Expression 2]

SRₘ₀=αₘ*SCₘ₁+βₘ*SCₘ₂+ . . . +εₘ (5)

In Mathematical Expression (5), the score SCm1 indicates, for example, an index related to stress, and the score SCm2 indicates, for example, an index related to concentration. As the stress score SCm1, for example, a stress index that is a ratio (LF/HF) of a high-frequency variation component (HF component) corresponding to the respiratory variation extracted from time-series data of the heart rate variation and a low-frequency component (LF component) corresponding to the blood pressure variation is used. Furthermore, for example, the concentration level is determined on the basis of the movement of the expression muscles, blinks, and changes in the line of sight and the face orientation from a captured image obtained by imaging a user, and the value of the concentration score SCm2 is made smaller as the concentration level is lower.

The coefficients αm and βm are preset coefficients indicating weights of the respective scores. Note that the coefficients indicating the weights, the scores, and the offset cm are set in advance so as to satisfy a predetermined condition, similarly to the case of calculating the physical power initial value.

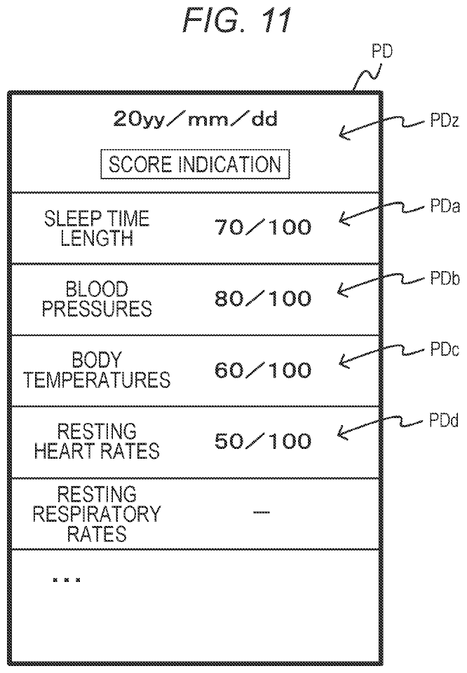

Note that in the power initial value calculation processing, the user may be notified of the score of each item. FIG. 11 illustrates an example of an initial value score display screen. For example, in a case where the optimum value of the sleep time length is 7 hours and the initial value of the sleep time length is 5 hours, the score of the initial value of the sleep time length is “0.7” from FIG. 9. Thus, a sleep time length PD1 on an initial value score display screen PD is indicated as “70/100”. Furthermore, in a case where the systolic blood pressure is 120 [mmHg] and the diastolic blood pressure is 80 [mmHg] as the initial values of the blood pressures, the initial values are determined to be within the range of the normal blood pressures from FIG. 10, and the score of the initial values of the blood pressures is “0.8”, so that a blood pressure PDb is indicated as “80/100”. Furthermore, for body temperatures PDc, resting heart rates PDd, and the like, scores are indicated. Note that an indication PDz is an initial value of the date.

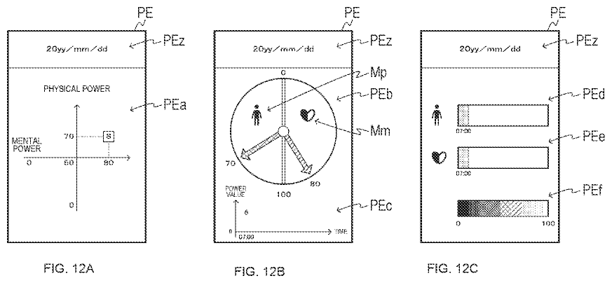

In the power initial value notification processing, the user is notified of the power initial values calculated by the server 40, that is, the physical power initial value SRpO and the mental power initial value SRm0. FIG. 12 illustrates an example of a power initial value notification screen. (a) of FIG. 12 illustrates, as an example, a case where on a power initial value notification screen PE, the temporal changes of the physical power and the mental power are indicated as a coordinate position change in a two-dimensional indication PEa, and the coordinate position “s” on the screen indicates the initial values of the physical power and the mental power. In (b) of FIG. 12, the initial values of the physical power and the mental power are indicated as ratio indications by, for example, the positions of hand pointers on a radial gauge indication PEb. Furthermore, the initial values of the physical power and the mental power are also indicated on a line graph indication PEc. (c) of FIG. 12 illustrates a case where the initial values of the physical power and the mental power are indicated as start position information of temporal progress status indications, for example, progress bar indications PEd and PEe, and the color or the luminance of the indications is set as specified in an indication PEf according to the power values. Note that a mark Mp in (b) and (c) of FIG. 12 indicates the physical power, and a mark Mm in (b) and (c) of FIG. 12 indicates the mental power, and the same applies to the drawings to be described later.

In the remaining power calculation processing, the remaining physical power and the remaining mental power for each user are calculated on the basis of the physical power initial value and the mental power initial value, the biological information acquired by the measurement device 20 and the user input information that is input on the terminal device 30 thereafter, and the like.

In the remaining power value calculation processing, a remaining physical power SRpr is calculated on the basis of, for example, Mathematical Expression (6).

[Mathematical Expression 3]

SRₚᵣ=SRₚᵢ−SCₚᵥ+Gₚ (6)

In Mathematical Expression (6), a remaining physical power SRpi is the previous remaining physical power, and in the first calculation of the remaining physical power value, the physical power initial value is used as the remaining physical power SRpi. A power change amount SCpv indicates a power change amount of the physical power according to the exercise strength during an interval period of updating the remaining power. Furthermore, a power recovery amount Gp indicates the physical power recovery amount during the interval period of updating the remaining power.

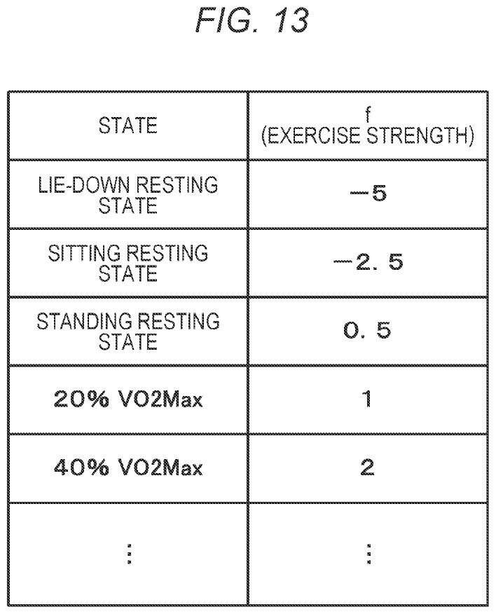

FIG. 13 illustrates an example of the power change amount of the physical power according to the exercise strength. For example, when a user is in a lie-down resting state, the power change amount is “−5”, and the physical power recovers by “5” during the interval period of updating the remaining power. When a user is in a sitting resting state, the change amount is “−2.5”, and the physical power recovers by “2.5” during the interval period of updating the remaining power. When the user is in a standing resting state, the power change amount is “0.5”, and the physical power is consumed by “0.5” during the interval period of updating the remaining power. When the exercise strength corresponds to the oxygen intake amount of “20% VO2Max”, the power change amount is “1”, and the physical power is consumed by “1” during the interval period of updating the remaining power. As described above, in the power change amount, the consumption of the physical power during the interval period of updating the remaining power is larger as the exercise strength is larger. Note that the power change amount of the physical power is not necessarily determined using the oxygen intake amount or the like as the exercise strength, and may be calculated using metabolic equivalents (METs), a ratio of the measured heart rate to the maximum heart rate, or the like.

The power recovery amount Gp is a physical power recovered on the basis of a nutritional value (for example, calories or the like) acquired by, for example, a meal or the like, and the power recovery amount may be, for example, input by a user, or the power recovery amount Gp may be automatically calculated from information of ingredients taken in by a user or the like that is input.

Furthermore, in the remaining power value calculation processing, a remaining mental power SRmr is calculated on the basis of, for example, Mathematical Expression (7).

[Mathematical Expression 4]

SRₘᵣ=SRₘᵢ−SCₘᵥ+Gₘ (7)

In Mathematical Expression (7), a remaining mental power SRmi is the previous remaining mental power, and in the first calculation of the remaining mental power value, the mental power initial value is used as the remaining mental power SRmi. A power change amount SCmv indicates a power change amount of the mental power according to stress during an interval period of updating the remaining power. Furthermore, a power recovery amount Gm indicates the mental power recovery amount during the interval period of updating the remaining power.

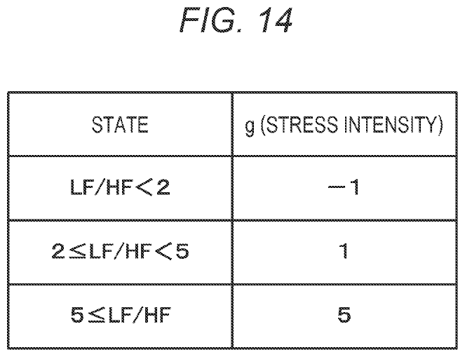

FIG. 14 illustrates an example of the power change amount of the mental power according to stress. As an index indicating stress, for example, the above-described stress index (LF/HF) is used. In a case where the stress index (LF/HF) is smaller than “2”, the power change amount is “−1”, and the mental power recovers by “1” during the interval period of updating the remaining power. In a case where the stress index (LF/HF) is “2” or larger and smaller than “5”, the power change amount is “1”, and the mental power is consumed by “1” during the interval period of updating the remaining power. Furthermore, in a case where the stress index (LF/HF) is “5” or larger, the power change amount is “5”, and the mental power is consumed by “5” during the interval period of updating the remaining power. As described above, in the power change amount, the consumption of the mental power during the interval period of updating the remaining power is larger as the stress is larger.

The power recovery amount Gm is a mental power recovered on the basis of a nutritional value (for example, calories or the like) acquired by, for example, a meal or the like, and the power recovery amount may be, for example, input by a user, or the power recovery amount Gm may be automatically calculated from information of ingredients taken in by a user or the like that is input.

Moreover, in the remaining power calculation processing, a user may be notified of the remaining power calculated every time when the update interval period elapses together with the elapsed time.

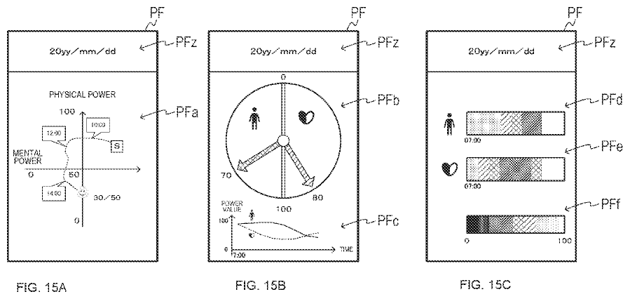

FIG. 15 illustrates an example of a remaining power notification. (a) of FIG. 15 illustrates, as an example, a case where on a remaining power notification screen PF, the temporal changes of the physical power and the mental power are indicated as a coordinate position change in a two-dimensional indication PFa, and the remaining power change from “s” indicating the power initial values on the screen is indicated as a trajectory on the two-dimensional indication PFa. Note that in FIG. 15, the positions at time points “10:00”, “12:00”, and “14:00” are indicated as distinguishable positions. Furthermore, the coordinate position of the end point (facial mark position) of the trajectory indicates the current remaining powers. In (b) of FIG. 15, the remaining powers of the physical power and the mental power are indicated by the positions of the hand pointers in a radial gauge indication PFb. Furthermore, the temporal changes of the remaining powers are indicated in a line graph indication PFc. Note that in the line graph indication PFc, a solid line indicates an example of a change in physical power, and a broken line indicates an example of a change in mental power. In (c) of FIG. 15, the progress in time of the physical power and the mental power is indicated in progress bar indications PFd and PFe, and the remaining power values are indicated by an indication color, a luminance level, a pattern, a shade, or the like in accordance with the progress in time.

In the activity support information notification processing, activity support information indicating an activity that can be performed with the remaining powers is generated. In the server 40, a physical power and a mental power necessary for performing an activity for a predetermined time length are recorded for each activity in the recording unit 43, for example, in a database. The processing unit 42 of the server 40 generates activity support information indicating an activity and an activity time length that are possible with the remaining powers from the calculated remaining powers, and the physical power and the mental power necessary for each activity, and transmits the activity support information from the communication unit 41 to the terminal device 30. The terminal device 30 notifies, from the notification unit 33, the user of the activity support information generated by the server 40.

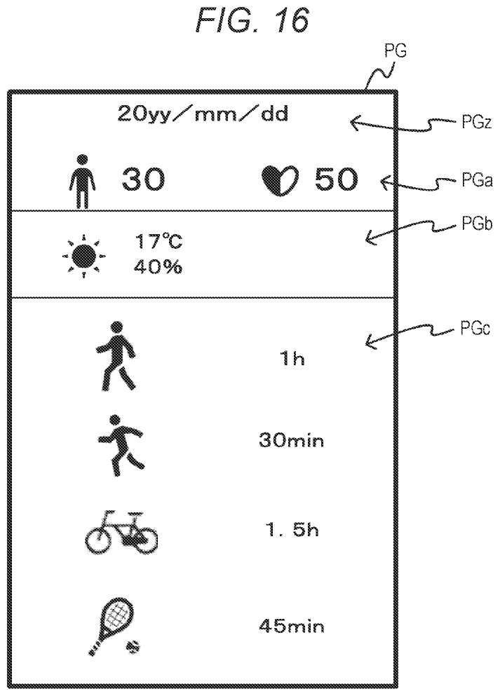

FIG. 16 illustrates an example of a notification of activity support information. On an activity support information notification screen PG, an indication PGa indicating a remaining physical power (for example, “30”) and a remaining mental power (for example, “50”) is provided. Furthermore, an indication PGc indicating possible activities and possible activity time lengths with the remaining powers, for example, walking for 1 hour, running for 30 minutes, cycling for 1.5 hours, playing tennis for 45 minutes, and the like are provided Note that an indication PGz indicates the date, and an indication PGb indicates the weather, the temperature, and the humidity at the user position.

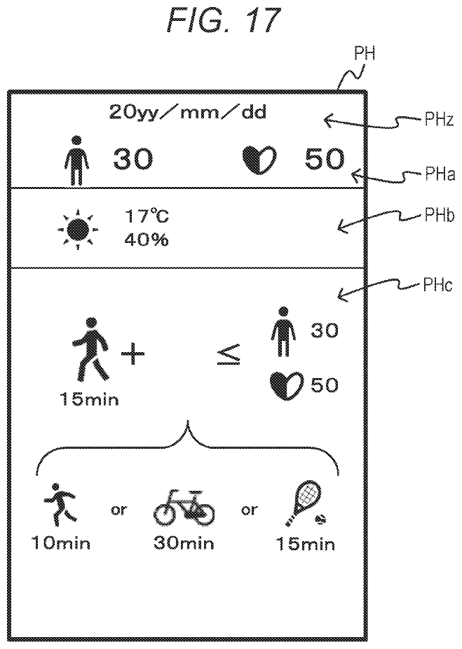

Furthermore, as the notification of activity support information, a user may be notified of an activity and activity time length that are further possible according to the selected activity. FIG. 17 illustrates an example of a notification of activity support information indicating an activity and activity time length that are further possible according to the selected activity. On an activity support information notification screen PH, for example, an indication PHc is provided and the indication PHc indicates that, for example, in a case where walking is selected and the activity time length is set to 15 minutes, when the physical power is “30” and the mental power is “50” or less, an activity that can be added is, for example, running for 10 minutes, cycling for 30 minutes, or playing tennis for 15 minutes. Note that an indication PHz indicates the date, and an indication PHb indicates the weather, the temperature, and the humidity at the user position.

Furthermore, in the activity support information notification processing, a user may be notified of a possible activity using map information. For example, the server 40 calculates a movable distance on the basis of the remaining powers and the exercise strength corresponding to the walking speed, and generates activity support information indicating a movable range ARa from the current location on the actual map.

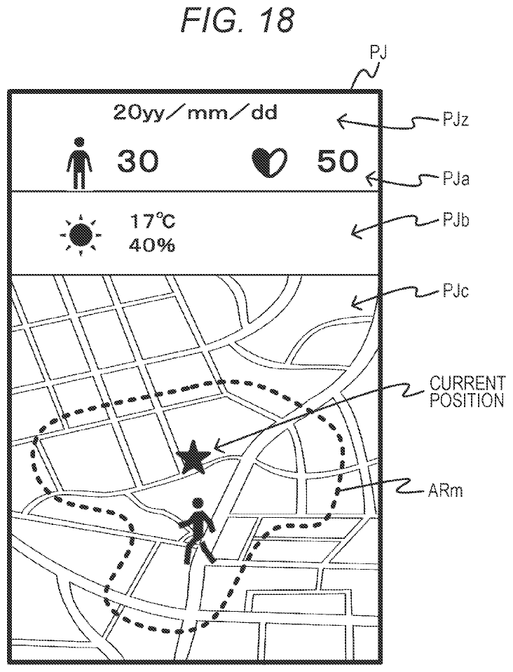

FIG. 18 illustrates an example of a notification of activity support information indicating a possible activity range in a case where walking is selected as an activity. On an activity support information notification screen PJ, an indication PJa indicating a remaining physical power (for example, “30”) and a remaining mental power (for example, “50”) is provided. Moreover, in an indication PJc, in a case where walking is selected, a movable range ARm from the current position as a reference is indicated on an actual map. Note that an indication PJz indicates the date, and an indication PJb indicates the weather, the temperature, and the humidity at the user position. Indication of a movable range using an actual map in this way enables easy selection of a spot within the movable range. Note that the spot (facility, store, or the like) may be a spot of a type specified by a user, or may be a spot of a type determined on the basis of the preference, the past activity history, and the like of the user recorded in the recording unit 43 or the like of the server 40.

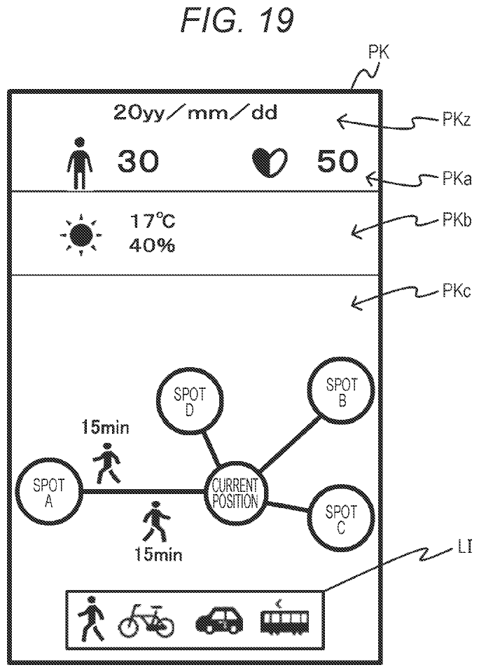

Furthermore, the processing unit 42 of the server 40 may generate, as the movable range notification information, a virtual map indicating a facility to which a user can move with remaining powers out of facilities of a type specified by the user or recommended facilities selected on the basis of the preference, the past activity history, and the like of the user. Note that the preference, past activity history, and the like of a user are recorded in the recording unit 43 or the like. FIG. 19 illustrates another example of a notification of activity support information indicating a possible activity range. On an activity support information notification screen PK, an indication PKa indicating a remaining physical power (for example, “30”) and a remaining mental power (for example, “50”) is provided. Moreover, in an indication PKc, for example, spots A to D to which a user can move with remaining powers are located radially from the current position as a virtual map. Moreover, in the indication PKc, the moving time length when the user moves to a facility is indicated. Since recommended facilities corresponding to the remaining powers are displayed in this way, a preferred facility can be easily selected without searching an actual map for a facility. Note that although FIG. 19 illustrates an example of a case where the moving means is “walking”, a list of icons LI indicating a plurality of moving means may be provided, and in a case where, for example, a user selects another moving means instead of “walking”, the remaining physical power and the remaining mental power when the selected moving means is selected, or a spot or the like to which the user can move with the remaining powers may be indicated. Furthermore, also in the case of FIG. 18, the list of icons LI indicating a plurality of moving means may be provided to notify a user of a movable range using an actual map according to the moving means selected by the user.

The powers required for an activity change according to the activity environment. Therefore, the server 40 may correct the exercise strength according to the activity environment on the basis of activity environment information acquired by the measurement device, activity environment information set by a user, activity environment information acquired from the Web, or the like. The activity environment information is information indicating a situation of the activity environment, and includes, for example, information such as the weather, the temperature, the humidity, the wind direction, the wind power, the steps and gradients in a movement route, and ease of traveling.

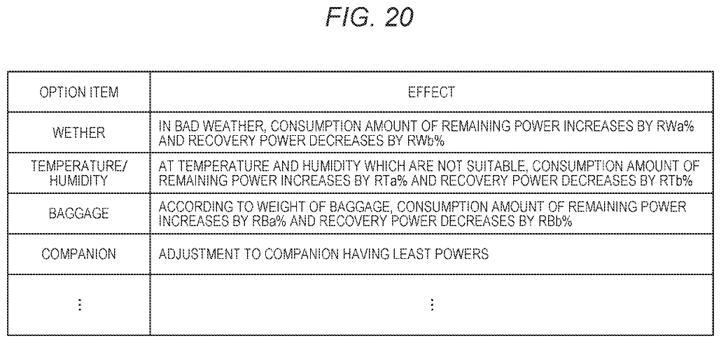

FIG. 20 illustrates an example of correction of the exercise strength based on the activity environment information. For example, in a case where weather is used as the activity environment information, the exercise strength is corrected such that powers are consumed more and recovery of the powers is less in bad weather.

Furthermore, in a case where temperature and humidity are used as the activity environment information, the exercise strength is corrected such that consumption of powers is more and the recovery of powers is less when the temperature is high or low compared to the temperature suitable for the activity, and when the humidity is high or low compared to the humidity suitable for the activity.

Moreover, since the power consumption is more in a case where a baggage is carried, the exercise strength is corrected such that the power consumption is more as the gravity of the baggage is more. Furthermore, in a case where there is a companion, it is necessary to perform an activity fitted to the companion, and thus, in generation of activity support information, correction is performed to adjust the activity time length to a companion having the least powers.

With correction of the exercise strength according to the activity environment information in this way, the activity support information indicating an activity, activity time length, and the like that are possible can be generated more accurately according to the remaining powers.

### 4. APPLICATION EXAMPLE

### 4-1. First Application Example

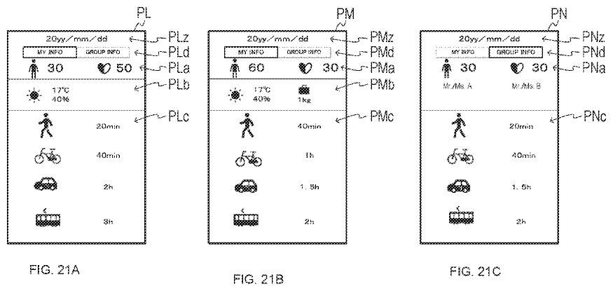

Next, an application example of the information processing system will be described. In a first application example, a case where the information processing system is applied to determination of an action during travel will be described. FIG. 21 illustrates an example of a remaining power notification of a user A and a user B traveling together.

An activity support information notification screen PL of the user A illustrated in (a) of FIG. 21 is provided with an indication PLa indicating a remaining physical power (for example, “30”) and a remaining mental power (for example, “50”), and an indication PLc indicating a possible activity and a possible activity time length with the remaining powers, for example, 20 minutes in a case where the moving means is walking, 40 minutes in a case where the moving means is a bicycle, 2 hours in a case where the moving means is a car, 3 hours in a case where the moving means is a train, or the like. Note that an indication PLz indicates the date, and an indication PLb indicates the weather, the temperature, and the humidity at the user position. Furthermore, an indication PLd indicating whether the activity is a group activity or an individual activity is provided, and in the figure, the indication PLd indicates that the notification is “MY INFO”, which is a notification in the case of an individual activity.

An activity support information notification screen PM of the user B illustrated in (b) of FIG. 21 is provided with an indication PMa indicating a remaining physical power (for example, “60”) and a remaining mental power (for example, “30”), and an indication PMc indicating a possible activity and a possible activity time length with the remaining powers, for example, 40 minutes in a case where the moving means is walking, 1 hour in a case where the moving means is a bicycle, 1.5 hours in a case where the moving means is a car, 2 hours in a case where the moving means is a train, or the like. Note that an indication PMz indicates the date, and an indication PMb indicates the weather, the temperature, and the humidity at the user position, and that the user possesses a baggage (1 Kg). Furthermore, an indication PLd indicating whether the activity is a group activity or an individual activity is provided, and in the figure, the indication PMd indicates that the notification is “MY INFO”, which is a notification in the case of an individual activity.

In a case where the user A and the user B are on an activity as a group, the processing unit 42 of the server 40 generates activity support information on the basis of the minimum values among the remaining powers of the users in the group. In (c) of FIG. 21, an example of an activity support information notification screen in a case where the user A and the user B are on an activity as a group is illustrated. On an activity support information notification screen PN, an indication PNa indicating that the minimum among the remaining physical powers is “30 (Mr./Ms. A)” and the minimum among the remaining mental powers is “30 (Mr./Ms. B)”. Furthermore, there is provided an indication PNc indicating a possible activity and a possible activity time length with the minimum values of the remaining physical power and the remaining mental power, for example, 20 minutes in a case where the moving means is walking, 40 minutes in a case where the moving means is bicycle, 1.5 hours in a case where the moving means is a car, 2 hours in a case where the moving means is a train, or the like. Note that the indication PLz indicates the date, and the indication PLd indicates that the notification is “GROUP INFO”, which is a notification in the case of a group activity. Furthermore, there is a user possessing a baggage in the group as illustrated in (b) of FIG. 21, and thus the exercise strength may be corrected according to the weight of the baggage, the activity and activity time length that are possible may be calculated on the basis of the corrected exercise strength, and the minimum values among the remaining physical powers and among the remaining mental powers, and the users may be notified of the activity and activity time length that are possible.

As described above, in a case where a group activity is performed, users are notified of an activity and an activity time length that are possible according to the remaining powers of users in the group, and thus, it is possible to prevent a user having less remaining powers or the like from being unable to perform the activity.

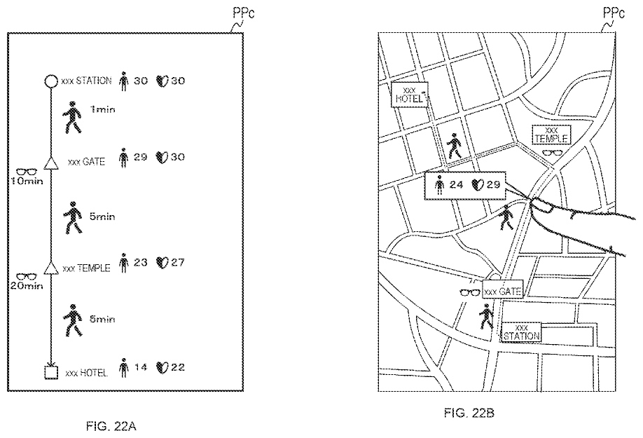

Next, a case where activity support information based on the remaining powers is generated using map information and a user is notified thereof is described as an example. The server 40 determines a route for using one or a plurality of facilities selected by a user, and calculates a moving time length according to the moving means. Moreover, the remaining powers at the time of arrival at a facility are calculated and included in the activity support information. FIG. 22 illustrates an example of a notification of activity support information generated on the basis of the remaining powers using map information. A support information notification indication PPc on the activity support information notification screen indicates, as an example, a case where a user travels from “xxx station” that is an activity start position to a spot (“xxx gate” and “xxx temple”) for sightseeing, and then moves to “xxx hotel” that is an activity end position. Note that on the activity support information notification screen, the date, weather, temperature, humidity, and the like may be indicated as described above.

In (a) of FIG. 22, a support information notification indication PPa for notifying a user of activity support information in a schedule format (flow format) is illustrated. The support information notification indication PPc indicates a remaining physical power “30” and a remaining mental power “30” at the activity start position. Furthermore, the support information notification indication PPc indicates that the time length required for moving from “xxx station” to “xxx gate” by walking is 1 minute, and the remaining powers expected when arriving at “xxx gate” include the remaining physical power of “29” and the remaining mental power of “30”.

The support information notification indication PPc indicates that the standard time length required for sightseeing at “xxx gate” is 10 minutes, the time length required for walking to “xxx temple” after the sightseeing is 5 minutes, and the remaining powers expected when arriving at “xxx temple” include the remaining physical power of “23” and the remaining mental power of “27”.

Furthermore, the support information notification indication PPc indicates that the standard time length required for sightseeing at “xxx temple” is 20 minutes, the time length required for moving to “xxx hotel” by walking after the sightseeing is 5 minutes, and the remaining powers expected when arriving at “xxx hotel” include the remaining physical power of “14” and the remaining mental power of “22”.

In (b) of FIG. 22, a support information notification indication PPc for notifying a user of activity support information in a map format is illustrated. On the support information notification indication PPc, an activity start position, an activity end position, spots, and a movement route are indicated on an actual map. Note that a broken line on the actual map is a movement route, and an icon indicates that the movement is performed by walking. Furthermore, the movement route may be indicated with an icon indicating, for example, movement is performed by walking as an icon animation that is indicated while moving the movement route.

Here, for example, when the user specifies a spot, an activity end position, or a movement route, remaining powers predicted at the instructed position are indicated. For example, in a case where a movement route is specified, it is indicated that the remaining powers expected at this point include the remaining physical power of “24” and the remaining mental power of “29”.

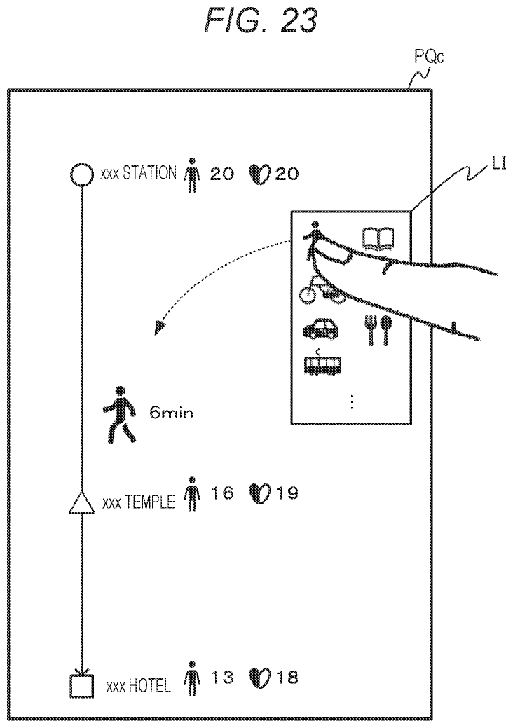

Note that the notification of the activity support information is not limited to notifying a user using an image as an example illustrated in FIG. 22, and a user may be notified of a movement route or expected remaining powers by sound, or a user may be notified when the remaining powers become lower than preset thresholds by an operation such as vibration. Furthermore, in the notification of the activity support information, only the support information notification indication may be indicated, or the date and the like may be also displayed as described above. Note that the same applies to FIGS. 23 to 26 to be described later.

As described above, the information processing system 10 notifies a user of, as activity support information, the remaining powers expected according to the activity plan, and thus the user can avoid an unreasonable activity.

Furthermore, the information processing system 10 can change the activity support information on the basis of a user instruction such that the expected remaining powers do not become less than a desired level. For example, in a case where the user desires an activity that makes the remaining physical power and the remaining mental power expected at the activity end position be 10 or more is assumed. If the remaining physical power is “20” and the remaining mental power is “20” at the activity start position, and the activities indicated in FIG. 22 are performed, the remaining physical power expected at the activity end position is less than “10”.

Thus, the user performs a change operation of deleting stopping by to some of the spots. FIG. 23 illustrates an example of the operation of changing the activity support information after the deletion of the stopping by to some spots. On a change operation screen PQc, for example, since the stopping by to the spot “xxx gate” has been deleted, the moving means from the spot “xxx station” to the spot “xxx temple” is set. Note that FIG. 23 illustrates an example of a case where the user selects “walking” as the moving means from the list of icons LI.

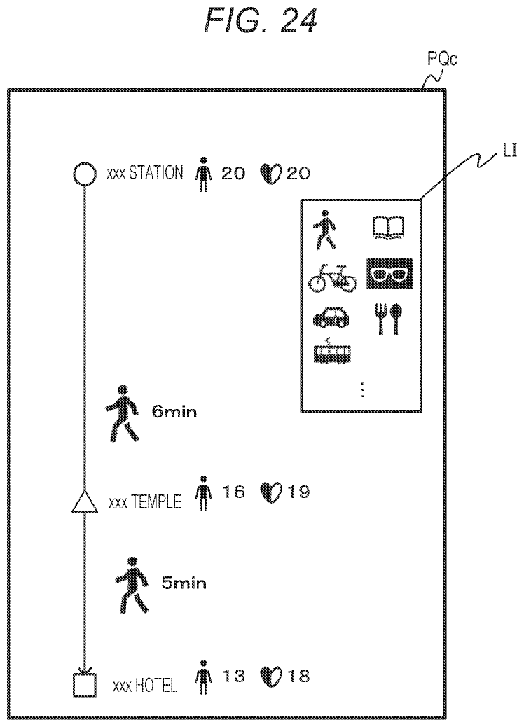

FIG. 24 illustrates an example of an operation of changing the activity support information after the moving means is selected. The change operation screen PQc illustrates a case where the stopping by to the spot “xxx gate” is deleted and “walking” is selected as the moving means. For example, in a case where the remaining physical power is “20” and the remaining mental power is “20” at the activity start position, and when the activities illustrated in FIG. 23 are performed with, the remaining powers expected at the activity end position include the remaining physical power of “13” and the remaining mental power of “18”, which are more than “10”. Here, if sightseeing at the spot “xxx temple” consumed the physical power “6” and the mental power “4”, the remaining physical power expected at the activity end position would be less than “10”. Therefore, the server 40 makes an icon of an activity, from the list of icons LI, that would make the remaining physical power expected at the activity end position less than “10”, that is, an icon indicating sightseeing (eyeglass mark) un-selectable on the change operation screen. Note that an icon that is indicated in reversed color in the list of icons LI (the background is black) indicates that the icon is un-selectable.

As described above, the activity that would make the remaining powers expected at the activity end position less than the desired value is set to be un-selectable, and thus the activity support information can be efficiently changed.

Furthermore, by including an activity that can recover powers in selectable activities, the activity support information can be generated in consideration of not only power consumption but also power recovery.

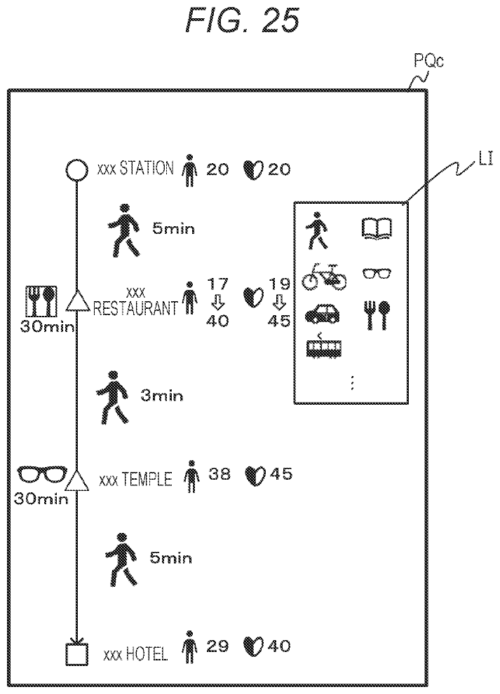

FIG. 25 illustrates an example of a change operation in a case where a spot where the powers can be recovered is selectable. On the change operation screen PQc, for example, in a case where the user selects to take a break at a spot “xxx restaurant” before the spot “xxx temple”, the server 40 calculates the remaining powers including the activity of taking a break at the spot “xxx restaurant”, and notifies the user of the remaining powers at the time of arrival and departure of the spot “xxx restaurant”. Note that in FIG. 25, for example, it is indicated that the remaining physical power is recovered from “17” at the time of arrival at “xxx restaurant” to “40”, and the remaining mental power is recovered from “19” to “45”. Since the powers have been recovered, the server 40 calculates the remaining powers expected at the activity end position in a case where sightseeing at the spot “xxx temple” is selected. Here, in a case, for example, where the remaining physical power is “29” and the remaining mental power is “40”, the remaining powers at the activity end position are “10” or more, and thus the icon indicating sightseeing in the list of icons LI is made selectable. Furthermore, in a case where the user selects sightseeing at the spot “xxx temple”, the server 40 notifies the user of the remaining physical power of “29” and the remaining mental power of “40” that are expected at the activity end position when the user selects sightseeing.

Furthermore, the server 40 may notify a user of a spot that is not away from the movement route, that matches the user's preference, and that can recover the powers on the basis of the preference, the past activity history, and the like of the user recorded in the recording unit 43 or the like and the activity start position, the activity end position, and the positions of spots, and make the spot selectable, so that the activity support information can be changed efficiently and effectively.

### 4-2. Second Application Example

In a second application example, a case where a user goes shopping will be described. For example, the server 40 generates activity support information indicating a position of a store of a type (for example, a supermarket, a convenience store, a drug store, or the like) selected by the user on the actual map. Moreover, the server 40 determines a store that a user can make a round trip on the basis of the biological information of the user and activity environment information, and switches the attribute of the store indication (for example, the size, color, shape, and the like of the store mark) so that the store that the user can make a round trip can be distinguished from other stores.

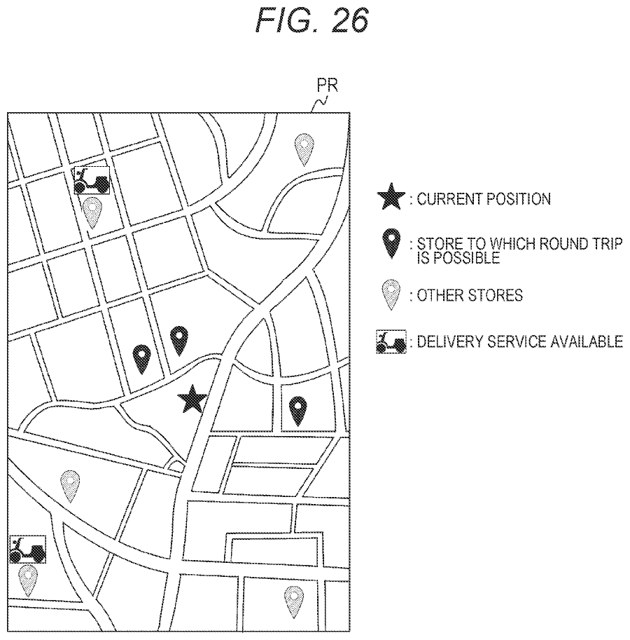

FIG. 26 illustrates an example of a notification of activity support information in a case where the user goes shopping. On an activity support information notification screen PR, a current position, stores that the user can make a round trip and other stores are indicated on the actual map. By thus notifying the user of the activity support information, the user can easily determine a store where the user can, for example, shop when the user is in a bad condition.

Furthermore, regarding the stores that the user cannot make a round trip, the attribute of the store indication may be changed such that it is possible to identify whether a delivery service from the store is available. In this case, even if the user is in a bad condition and cannot go to a store, the user can obtain a desired product using the delivery service.

### 4-3. Third Application Example

In a third application example, a case where a managing person (guardian) requests a managed person (protected person) to shop or the like will be described. In this case, the measurement device 20 and the terminal device 30-1 are possessed by the managed person, and the managing person uses the terminal device 30-2.

The managing person instructs the destination to the managed person, and transmits destination information to the server 40 via the terminal device 30-2.

The terminal device 30-1 possessed by the managed person captures and displays a surrounding image of the managed person. Furthermore, the terminal device 30-1 transmits the surrounding image to the terminal device 30-2. Moreover, the terminal device 30-1 transmits information indicating the current position of the managed person to the terminal device 30-2 possessed by the managing person and the server 40.

The measurement device 20 acquires the biological information of the managed person and transmits the biological information to the server 40 or to the server 40 via the terminal device 30-1.

The server 40 generates route guidance information on the basis of the destination information and the current position of the managed person. Furthermore, the server 40 calculates the remaining powers on the basis of the biological information of the managed person. The server 40 transmits activity support information indicating the remaining powers of the managed person and route guidance information to the terminal device 30-1. Furthermore, the server 40 transmits activity support information indicating the remaining powers of the managed person to the terminal device 30-2. In a case where advice information is generated in response to the notification of the activity support information to the managing person, the terminal device 30-2 transmits the generated advice information to the terminal device 30-1.

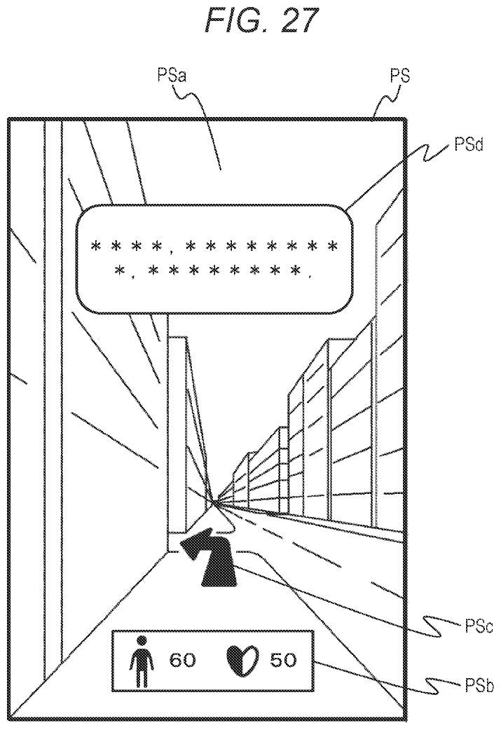

FIG. 27 illustrates an example of a notification of activity support information to a managed person who has been requested to shop or the like. An activity support information notification screen PS is provided with an indication PSa of a surrounding image and an indication PSb indicating a physical power and a mental power of the managed person. Moreover, an indication PSc of route guidance information and an indication PSd indicating advice information from the managing person are provided.

Therefore, the managed person can easily reach the destination using the route guidance information. Furthermore, the managed person can check his/her remaining powers and perform an activity with reference to advice from the managing person.

Furthermore, the managing person can grasp the remaining powers and the surrounding situation of the managed person in real time on the basis of the activity support information of which the managing person is notified through the terminal device 30-2. Furthermore, the managing person can notify, from the terminal device 30-2, the managed person of appropriate advice according to the remaining powers of the managed person and the surrounding situation. Moreover, in a case where the managed person is notified of the activity support information through the terminal device 30-2, indication that enables easy identification that the remaining powers are low when the remaining powers of the managed person are less than a preset threshold (warning display, color-coded display, or the like) can allow the managing person to take a measure such as going to meet the managed person or the like.

### 4-4. Fourth Application Example

In a fourth application example, a case where a managing person manages a plurality of users (employees) to be managed will be described. For example, each of the employees possesses the measurement device 20 or the measurement device 20 and the terminal device 30-1, and the managing person who manages the employees uses the terminal device 30-2. The measurement device 20 of each employee acquires the biological information or the like and transmits the biological information to the server 40 or to the server 40 via the terminal device 30-1. The server 40 calculates the remaining physical power and the remaining mental power for each employee. The server 40 transmits a remaining power list information indicating the calculated remaining physical powers and the remaining mental powers of the employees to the terminal device 30-2 as activity support information at a predetermined timing or in response to a request from the managing person.

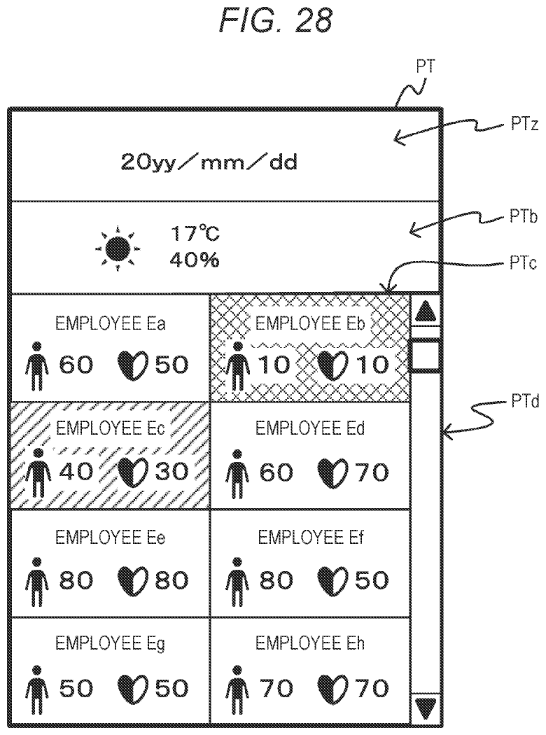

FIG. 28 illustrates an example of a notification of the remaining power list information. A remaining power list information notification screen PT is provided with an indication PTc in which the remaining physical powers and the remaining mental powers of the employees are listed in blocks. Furthermore, in a case where the number of employees is large, for example, a knob or an arrow button of a scroll bar PTb is operated to switch blocks to be displayed. Furthermore, an attribute of the block (for example, the color, the brightness of the background, or the like) is set according to the remaining powers. Therefore, it is possible to easily determine an employee or the like with less remaining powers on the basis of the attribute of the block. For example, according to the indication of FIG. 28, the managing person can easily determine that the remaining powers of an employee Ec are low and the remaining powers of an employee Eb are very low. Note that on the remaining power list information notification screen PT, an indication PTz indicates the date, and an indication PTb indicates the weather, the temperature and the humidity in the office.

Furthermore, the server 40 calculates the remaining physical power and the remaining mental power every day and every predetermined time length, and calculates temporal changes, for example, daily change amounts of the remaining powers or changes of the remaining powers within a day for each employee. Moreover, in response to a request from the terminal device 30-2 of the managing person, the server 40 outputs activity support information including power history information indicating the daily change amounts of the remaining powers and the like of a specified employee.

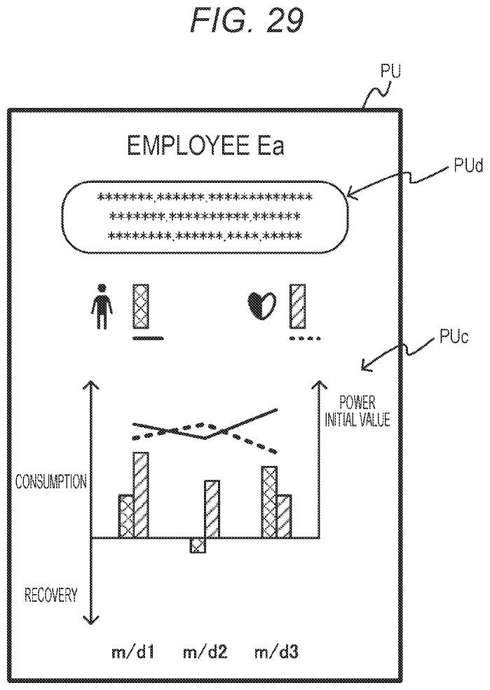

FIG. 29 illustrates an example of the power history information of a specified employee. A power history information notification screen PU is provided with an indication PUc indicating a history of the remaining powers and the power initial values of an employee Ea. Note that in the figure, the total amounts of the remaining physical power and the remaining mental power on each day in the period from d1 of m-month to d3 of m-month are indicated by a bar graph, and the transition of the initial values of the physical power and the remaining mental power is indicated by line graphs.

As described above, the remaining power history information notification screen PU enables grasping of power initial values and the changes in the remaining powers of a specified employee, and thus the managing person can generate appropriate advice information for the employee so as to prevent occurrence of physical disorder and mental disorder.

Furthermore, in a case where the terminal device 30-1 of the specified employee is notified of the remaining power history information, provision of an indication PUd of advice information from the managing person on the remaining power history information notification screen PU can enable physical condition management or the like with reference to the advice information.

Furthermore, the managing person can grasp the remaining powers of each employee, and thus the managing person can assign tasks according to the remaining powers. For example, the server 40 generates activity support information indicating an activity (task) and an activity time length that are possible with the remaining powers calculated for each employee. Furthermore, the server 40 outputs the activity support information of the specified employee in response to a request from the terminal device 30-2 of the managing person.

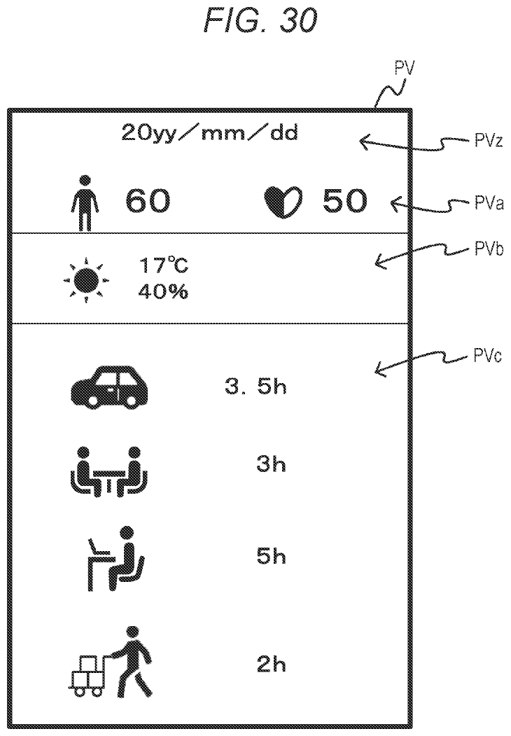

FIG. 30 illustrates an example of a notification of activity support information to a specified employee. On an activity support information notification screen PV, an indication PVa indicating a remaining physical power (for example, “60”) and a remaining mental power (for example, “50”) is provided. Furthermore, there is provided an indication PVc indicating that the possible activity and the possible activity time length with the remaining powers is, for example, a sales activity using a car for 3.5 hours, a meeting for 3 hours, desk work for 5 hours, a conveyance work for 2 hours, and so on. Note that an indication PVz indicates the date, and an indication PVb indicates the weather, the temperature, and the humidity at the user position.

In this way, the possible activity and the possible activity time length for a specified employee can be grasped, so that it is possible to efficiently perform various tasks by assigning tasks, for example, using the activity support information of each employee.

The series of processing steps described in the specification can be performed by hardware, software, or a combination of both. In the case of performing processing by software, a program in which a processing sequence is recorded is installed in a memory in a computer incorporated in dedicated hardware and executed. Alternatively, the program can be installed in and executed by a general-purpose computer capable of performing various types of processing.

For example, the program can be recorded in advance in a hard disk, a solid state drive (SSD), or a read only memory (ROM) as a recording medium. Alternatively, the program can be temporarily or permanently stored (recorded) in a removable recording medium such as a flexible disk, a compact disc read only memory (CD-ROM), a magneto optical (MO) disk, a digital versatile disc (DVD), a Blu-ray Disc (BD) (registered trademark), a magnetic disk, or a semiconductor memory card. Such a removable recording medium described above can be provided as so-called package software.

Furthermore, in addition to installing the program from the removable recording medium to the computer, the program may be transferred from a download site to the computer wirelessly or by wire via a network such as a local area network (LAN) or the Internet. The computer may receive the program thus transferred and install the program in a recording medium such as a built-in hard disk.

Note that the effects described in the present specification are merely examples and effects are not limited thereto, and there may be additional effects that are not described. Furthermore, the present technology should not be construed as being limited to the embodiment of the technology described above. The embodiment of this technology discloses the present technology in the form of exemplification, and it is obvious that those skilled in the art can make modifications and substitutions of the embodiments without departing from the gist of the present technology. That is, in order to determine the gist of the present technology, the claims should be taken into consideration.

Furthermore, the information processing device according to the present technology can also have the following configurations.

(1) An information processing device including: a power calculation unit configured to calculate a physical power and a mental power by using biological information of a user; and an information generation unit configured to generate activity support information for the user on the basis of the physical power and the mental power calculated by the power calculation unit.

(2) The information processing device according to (1), in which the activity support information indicates an activity and an activity time length that are possible on the basis of the physical power and the mental power of the user.

(3) The information processing device according to (2), in which the activity support information further indicates an activity capable of recovering the physical power or the mental power.

(4) The information processing device according (2) or (3), in which when any activity is selected from the activity that is possible, the information generation unit indicates, in the activity support information, an activity and an activity time length that are newly possible on the basis of the physical power and the mental power after the selected activity is performed.

(5) The information processing device according to any one of (2) to (4), in which the information generation unit calculates the activity time length using activity environment information.

(6) The information processing device according to any one of (1) to (6), in which the information generation unit generates the activity support information for the user and another person different from the user, on the basis of the physical power and the mental power calculated by the power calculation unit and the physical power and the mental power of the other person.

(7) The information processing device according to (6), in which the information generation unit generates the activity support information on the basis of a minimum value among the physical powers of the user and the other person and a minimum value among the mental powers of the user and the other person.

(8) The information processing device according to any one of (1) to (7), in which the power calculation unit calculates the physical power using an index related to a physical activity based on the biological information, and calculates the mental power using an index related to a mental activity based on the biological information.

(9) The information processing device according to any one of (1) to (8), in which the power calculation unit calculates initial values of the physical power and the mental power by using biological information when the user is in a best condition and the biological information of the user acquired at a start timing.

(10) The information processing device according to (9), in which the power calculation unit updates the physical power and the mental power using biological information of the user acquired after the start timing.

(11) The information processing device according to (10), in which the activity support information indicates temporal changes of the physical power and the mental power in a two-dimensional indication or a ratio indication, or as changes of an attribute in a temporal progress status indication.

(12) The information processing device according to (1), in which the activity support information indicates a possible activity on the basis of the physical power and the mental power using a map.

(13) The information processing device according to (12), in which the activity support information indicates, on the map, a route of an activity that is possible on the basis of the physical power and the mental power.

(14) The information processing device according to (13), in which the activity support information is for notification of the physical power and the mental power at a specified position of the route.

(15) The information processing device according to (13) of (14), in which the activity support information is for notification of the route with the icon moving on the map.

(16) The information processing device according to (1), in which the activity support information includes movement route information and instruction information from a managing person who manages the user.

(17) The information processing device according to (1), in which the activity support information indicates a list of the physical powers and the mental powers of management target users calculated by the power calculation unit.

(18) The information processing device according to (17), in which the information generation unit includes, in the activity support information, power history information indicating temporal changes of the physical power and the mental power of at least one of the management target users specified by a managing person.

### REFERENCE SIGNS LIST

10 Information processing system
20 Measurement device
21, 31 Input unit
22 Sensor unit
23, 33 Notification unit
24, 34, 43 Recording unit
25, 35, 41 Communication unit
26 Control unit
30, 30-1, 30-2 Terminal device
36, 42 Processing unit
40 Server
50 Network

1. An information processing device comprising:
a power calculation unit configured to calculate a physical power and a mental power by using biological information of a user; and
an information generation unit configured to generate activity support information for the user on a basis of the physical power and the mental power calculated by the power calculation unit.

2. The information processing device according to claim 1, wherein the activity support information indicates an activity and an activity time length that are possible on a basis of the physical power and the mental power of the user.

3. The information processing device according to claim 2, wherein the activity support information further indicates an activity capable of recovering the physical power or the mental power.

4. The information processing device according to claim 2, wherein when any activity is selected from the activity that is possible, the information generation unit indicates, in the activity support information, an activity and an activity time length that are newly possible on a basis of the physical power and the mental power after the selected activity is performed.

5. The information processing device according to claim 2, wherein the information generation unit calculates the activity time length using activity environment information.

6. The information processing device according to claim 1, wherein the information generation unit generates the activity support information for the user and another person different from the user, on a basis of the physical power and the mental power calculated by the power calculation unit and the physical power and the mental power of the other person.

7. The information processing device according to claim 6, wherein the information generation unit generates the activity support information on a basis of a minimum value among the physical powers of the user and the other person and a minimum value among the mental powers of the user and the other person.

8. The information processing device according to claim 1, wherein the power calculation unit calculates the physical power using an index related to a physical activity based on the biological information, and calculates the mental power using an index related to a mental activity based on the biological information.

9. The information processing device according to claim 1, wherein the power calculation unit calculates initial values of the physical power and the mental power by using biological information when the user is in a best condition and the biological information of the user acquired at a start timing.

10. The information processing device according to claim 9, wherein the power calculation unit updates the physical power and the mental power using biological information of the user acquired after the start timing.

11. The information processing device according to claim 10, wherein the activity support information indicates temporal changes of the physical power and the mental power in a two-dimensional indication or a ratio indication, or as changes of an attribute in a temporal progress status indication.

12. The information processing device according to claim 1, wherein the activity support information indicates a possible activity on a basis of the physical power and the mental power using a map.

13. The information processing device according to claim 12, wherein the activity support information indicates, on the map, a spot and a route of an activity that are possible on a basis of the physical power and the mental power.

14. The information processing device according to claim 13, wherein the activity support information is for notification of the physical power and the mental power at a specified position of the route.

15. The information processing device according to claim 13, wherein the activity support information is for notification of the route with an icon moving on the map.

16. The information processing device according to claim 1, wherein the activity support information includes movement route information and instruction information from a managing person who manages the user.

17. The information processing device according to claim 1, wherein the activity support information indicates a list of the physical powers and the mental powers of management target users calculated by the power calculation unit.

18. The information processing device according to claim 17, wherein the information generation unit includes, in the activity support information, power history information indicating temporal changes of the physical power and the mental power of at least one of the management target users specified by a managing person.

19. An information processing method comprising:
calculating, by a power calculation unit, a physical power and a mental power by using biological information of a user; and
generating, by an information generation unit, activity support information for the user on a basis of the physical power and the mental power calculated by the power calculation unit.

20. A program for causing a computer to perform activity support for a user by causing the computer to:
calculate a physical power and a mental power by using biological information of the user; and
generate activity support information for the user on a basis of the physical power and the mental power that are calculated.
